(19) United States
(12) Patent Application Publication
(10) Pub. No.: US 2015/0150652 A1
(43) Pub. Date: Jun. 4, 2015

(54) DENTAL IMPLANT

(71) Applicants: Salvatore BELLINVIA, (US); Cristiano VETTORI, (US); Erica VETTORI, (US); Carolina MALVAGNA, (US)

(72) Inventor: Cristiano Vettori, Pordenone (IT)

(21) Appl. No.: 14/411,567

(22) PCT Filed: Jul. 18, 2013
(86) PCT No.: PCT/EP2013/002140

§ 371 Date: Dec. 29, 2014

(30) Foreign Application Priority Data
Jul. 20, 2012 (IT) GE2012A000072

(51) Int. Cl. A61C 8/02; A61C 19/06; A61M 31/00; A61C 8/00; A61J 7/00

(52) U.S. Cl. CPC A61C 8/0006; A61C 8/0022; A61C 8/0068; A61J 7/0092; A61M 31/002; A61C 19/063

(57) ABSTRACT

Dental implant including a threaded shaft fitted with external threads (1) and a central tubular passage (2) fitted with an internal threaded portion (3), a fixing screw (5), complementary to it, which supports a dental crown or any other type of connection (6), and, in the end portion of the central tubular passage (2), there is a housing (7) to contain the medicament.

This invention concerns a dental implant. Various types of dental implants of differing forms are already known, for instance, with prosthetic connection and cylindrical or conical body. The implants mostly used are the endoseal type that have passed a series of clinical tests carried out following specific international protocols. The material generally used for said implants is titanium, a material that is able to create with a certain facility the union between the same implant and the bone (osseointegration).

More precisely, by osseointegration, one means the stable and lasting union between bone and implant that is able to allow the masticatory function without any complications. If, instead, osseointegration is not achieved, one must speak of fibro-integration that may mean a total failure, in which a stable bone-implant union is not created straight away or a partial failure in which the implant works without creating any problems only for a certain period of time. Among the most widespread problems that can be verified using the known implants, there is peri-implantitis which is an inflammation of the zone surrounding the implant, for which a tissue of defence is formed (the so called granulation tissue) in case of bacterial contamination of the implant surface. This tissue, in the most serious cases, may even come to replace the bone and to provoke osteomyelitis with consequent loss of the implant in a few weeks. Implants that lose osseointegration due to infectious causes have micro flora made up of spirochetes, fusobacteria and Gram negative rods and have an etiopathogenesis tightly correlated to the specific anatomical and histological morphostructure of the same tissues involved in the integration, to the superficial morphology of the fixtures and the precision of coupling of the implant components. Such particular conditions of the implant site mean that the principal etiological factor of peri-implant diseases is really that of infection.

Microbiological and clinical research has shown that an effective control of bacterial plaque reduces the possibility of peri-implant disease just as for periodontal disease. Until today, studies to overcome such problems have been concentrated on the external surface of implants using particular coatings able to contrast, at least partly, the occurrence of precocious and delayed bacterial infections, but still today it is necessary to place a pharmacological therapy in both a general and local way alongside the surgical therapy.

The subject of this invention is the realization of an implant made according to the known criteria, but that inside of which there is an empty space, of varying dimensions and shape, also identifiable as a prolongation of the receptive housing of the fixing screw or fixation of the superstructures or in a more apical or crown seating, created for this purpose, definable as a reservoir or housing, that communicates through one or more holes of various diameter with the external surface of the same implant in correspondence of any area of the smooth or rough surface (apical part, crown part, threaded part or any portion whatsoever in contact with the bone or mucous surfaces or of interface with the superstructure).

The communication of the aforesaid reservoir with the peri-implant environment can also be made by capillarity through the inside threading of the implant corresponding to the seat of insertion of the superstructure screw, with possible capillary diffusion of the substance directly inserted in the zone of implant emergency. Inside the aforesaid reservoir, one places a “tablet” made up of a disinfectant/antiseptic and/or an anti-inflammatory and/or an antibiotic/chemotherapy medicament and/or a factor stimulating bone regeneration and/or any medicine or derivate (for instance silver salts) suitable to encourage osseointegration and to contrast precocious and delayed infection. Such substance can be coated by a casing that determines a controlled release of it. This invention is therefore an implant able to resolve in a meaningful way the problems listed above through prevention and treatment of infections, through release of medicine from the interior of the same implant. In such way, the release of a disinfectant and/or antimicrobial is able to contrast bacterial contamination and an inflammatory reaction.

Furthermore, a growth factor may be used to accelerate the process of osseointegration.

The invention is further clarified through illustration of the exemplary solution in the attached drawings, in which:

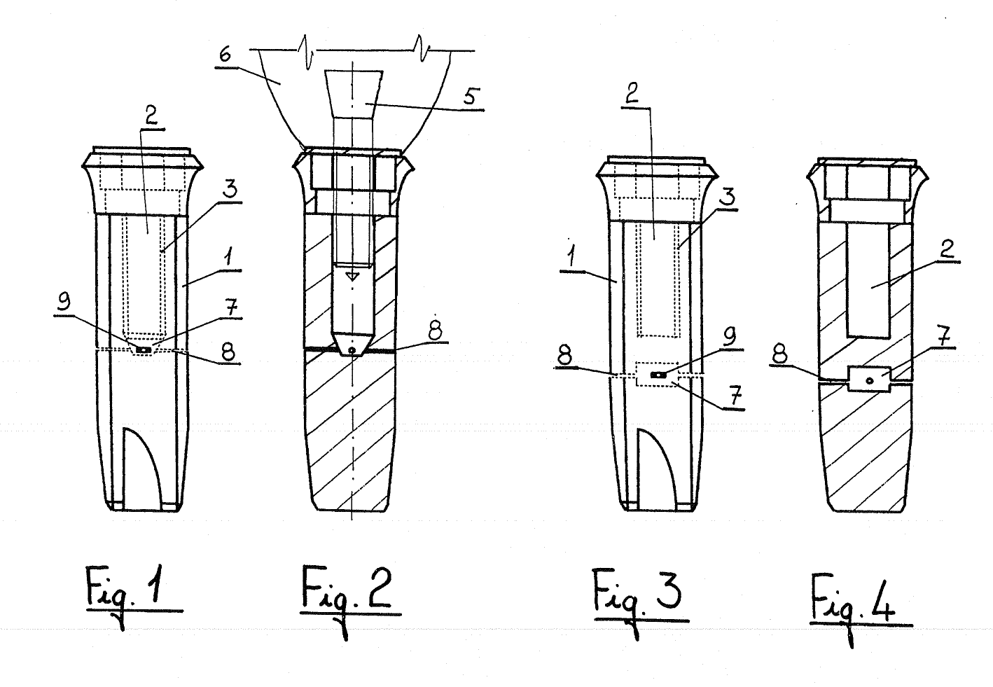

FIG. 1 illustrates a side view of the implant subject of this invention according to a first exemplary solution;

FIG. 2 illustrates a central cross-section of the implant in FIG. 1;

FIG. 3 illustrates a side view of the implant subject of this invention according to a further exemplary solution;

FIG. 4 illustrates a central cross-section of the implant in FIG. 3.

The dental implant in this invention includes a threaded shaft having external threads 1 endowed with a central passage 2, with an internal portion 3, also threaded, a fixing screw 5, complementary to said internal threaded portion, including a truncated-conical/cylindrical section adapted to receive a dental crown or an attachment 6 on it.

In the terminal part of the internally threaded portion there is a housing 7, of any shape (for instance inverted truncated-conical, cylindrical or spherical), that is connected with the external part of the same implant in contact with the bone, where infectious breeding grounds can be formed, through one or more connection canaliculi 8.

Inside the housing 7, a medicinal product is inserted, for instance, in the form of a tablet 9, which may be a disinfectant or an anti-inflammatory or an antibiotic/chemotherapy medicament and even a product stimulating bone regeneration or any medicine or derivate suitable to encourage osseointegration and to contrast precocious or delayed peri-implant infection.

Said tablet may be coated with a casing in such way as to determine a controlled release.

Said canaliculi can be of any convenient conformation in comparison to the axis of the implant so as to encourage the conduction of the medicament, where necessary.

If the tablet is totally consumed or it is necessary to replace the medicinal substance, it is possible to unscrew the fixing screw 5 and to insert the most opportune medicament and/or growth factor or any other useful product in the housing in that the latter is accessible through the aforesaid central passage 2.

There are many advantages of this system:

at the same time as the insertion of the implant we can obtain the release of a disinfectant and/or of an antimicrobial able to contrast bacterial contamination and an inflammatory reaction and/or a growth factor to accelerate the osseointegration;

at any later moment, after having unscrewed the superstructure of the same implant, one may insert the medicament or growth factor or any useful material in the reservoir;

the surgical procedure for insertion of the implant is not modified;

with the presence of the “tablet” one preventively intervenes on the possible complications before the highlighting of a radiological clinical situation with the result of an easier control of an infection/inflammation through a smaller quantity of active ingredient;

the waiting times for osseointegration are reduced, and at the same time the risks connected with an immediate load are reduced and secondary stability (stability in the middle/long term) is encouraged;

there is not the drawback of the quality-quantity deterioration of the active ingredient during the insertion of the implant in the bone;

the mechanical resistance of the implant is not jeopardized;

the external surface of the implant, whatever it may be, is unchanged;

at an industrial level, the production methodology of the same implant is not changed in a significant way, the holes being easy to make with innumerable techniques.

In an alternative solution illustrated in FIGS. 3 and 4, the housing 7 containing the medicinal tablet 9 is separated and independent from the central passage 2. In such case, the tablet containing the active ingredient is inserted inside the implant at the time of its realization or through the canaliculi of connection 8, before the insertion of the same implant in the tissue.

1. Dental implant comprising:
a threaded shaft fitted with external threads and central tubular passage fitted with an internal threaded portion, and
a fixing screw, complementary to the internal threaded portion it, which supports a dental connection, and
wherein an in e end portion of the central tubular passage includes there a housing ( )adapted to contain a tablet of at least one of the following:
anti-infective medicament,
anti-inflammatory medicament,
anti-bacterial medicament,
a medicament for stimulating tissue regeneration, and
a tablet containing a substance that encourages bone growth,
wherein the tablet is covered by a casing so as to facilitate a controlled release of the at least one of the medicament and the substance encouraging bone growth.

2. Dental implant according to claim 1, wherein said housing has an inverted truncated conical shape.

3. Dental implant according to claims 1 and 2, wherein said housing has at least one canaliculi connecting the housing with an external portion of the implant itself.

4. Dental implant according to claim 3, wherein the canaliculi connecting the external portion of the implant itself are longitudinal with respect to a vertical axis of the implant.

5. Dental implant according to claim 3, wherein the canaliculi connecting the external portion of the implant descend from the housing towards the external portion of the implant itself.

6-9. (canceled)
